(19) United States
(12) Patent Application Publication
(10) Pub. No.: US 2023/0201416 A1
(43) Pub. Date: Jun. 29, 2023

(54) IMPLANT MAGNESIUM ALLOY, BONE FIXTURE, METHOD OF MANUFACTURING IMPLANT MAGNESIUM ALLOY, AND METHOD OF MANUFACTURING BONE FIXTURE DEVICE

(71) Applicant: NATIONAL UNIVERSITY CORPORATION KUMAMOTO UNIVERSITY, Kumamoto (JP)

(72) Inventor: Yoshihito KAWAMURA, Kumamoto (JP)

(21) Appl. No.: 17/919,986

(22) PCT Filed: Apr. 21, 2021
(86) PCT No.: PCT/JP2021/016870

§ 371 Date: Oct. 19, 2022

(30) Foreign Application Priority Data
Apr. 21, 2020 (JP) 2020-075232

(51) Int. Cl. A61L 27/04; A61L 31/02; C22C 1/02; C22C 1/04; C22F 1/06; B21C 23/00; B22F 3/20

(52) U.S. Cl. CPC A61L 27/047; A61L 31/022; C22C 1/02; C22C 1/04; C22F 1/06; B21C 23/002; B22F 3/20; A61L 2430/02

(57) ABSTRACT

Provided is an implant magnesium alloy having corrosion resistance, mechanical strength, ductility at the same time. In one aspect of the present invention, an implant magnesium alloy contains: x at % of Zn; a total of y at % of at least one element of Ca and Sr; and the balance of Mg and inevitable impurities. x and y satisfy formulae 1 and 2:

0.15≤x≤1.5 (Formula 1)

0.5≤y≤1.5. (Formula 2)

### CROSS REFERENCE TO RELATED APPLICATION

This patent application claims the benefit and priority of Japanese Patent Application No. 2020-075232 filed on Apr. 21, 2020, the disclosure of which is incorporated by reference herein in its entirety as part of the present application.

### TECHNICAL FIELD

The present invention relates to an implant magnesium alloy, a bone fixture, a method of manufacturing an implant magnesium alloy, and a method of manufacturing a bone fixture.

### BACKGROUND ART

Stainless steel and titanium alloys are used for bone fixtures (such as plates and screws) used for therapy for bone fracture. Such alloys are not absorbed into the body, and hence permanently remains in the body unless the bone fixture is extracted by re-operation. Thus, materials for a bone fixture are desired to have a property of being absorbed to the body and have biological affinity.

As materials having biological absorbency and biological affinity, polylactic acid and magnesium alloys have been put into practice. WO 2016/038892 discloses the related art. Note that polylactic acid is a polymer in which lactic acid is polymerized by ester binding and connected long.

However, the mechanical strength of polylactic acid is insufficient for use as a bone fixture. A magnesium alloy does not have corrosion resistance sufficient for use as a bone fixture, and the mechanical strength is insufficient.

### SUMMARY OF INVENTION

### Problems to be Solved

One aspect of the present invention has an object to provide an implant magnesium alloy having both corrosion resistance and mechanical strength, a bone fixture, a method of manufacturing an implant magnesium alloy, or a method of manufacturing a bone fixture.

### Solution to Problem

Various aspects of the present invention are described below.

[1] An implant magnesium alloy, including: x at % of Zn; a total of y at % of at least one element of Ca and Sr; and the balance of Mg and inevitable impurities, in which x and y satisfy formulae 1 and 2 below.

0.15≤x≤1.5 (preferably 0.2≤x≤1.0) (Formula 1)

0.5≤y≤1.5 (preferably 0.75≤x≤1.25)(Formula 2)

[2] The implant magnesium alloy according to item [1], in which the magnesium alloy contains a at % of Mn, where a satisfies formula 3 below.

0.01≤a≤0.08 (preferably 0.02≤a≤0.05) (Formula 3)

[3] The implant magnesium alloy according to item [1] or [2], wherein the magnesium alloy contains z at % of RE (rare-earth element), wherein z satisfies formula 4 below.

0≤z≤0.2 (Formula 4)

[4] The implant magnesium alloy according to any one of items [1] to [3], wherein:

the magnesium alloy comprises a plurality of a-Mg grains; and

the plurality of a-Mg grains have an average grain diameter of 0.8 μm or more and 2.5 μm or less.

[5] The implant magnesium alloy according to any one of items [1] to [4], wherein the magnesium alloy comprises at least one of compounds of Mg₂Ca and Mg₁₇Sr₂.

[6] The implant magnesium alloy according to item [5], wherein the at least one compound has an average grain diameter of 0.07 μm or more and 0.29 μm or less.

[7] The implant magnesium alloy according to any one of items [1] to [6], wherein the magnesium alloy does not have a compound of Mg₆Ca₂Zn₃ in a constituent phase determined by X-ray diffraction.

[8]. The implant magnesium alloy according to any one of items [1] to [7], wherein the magnesium alloy does not contain an unavoidable amount or more of Al.

[9] A bone fixture, comprising the implant magnesium alloy according to any one of items [1] to [8].

[10] A method of manufacturing an implant magnesium alloy, comprising the steps of:

(a) rapidly solidifying a molten metal of a magnesium alloy to manufacture a plurality of rapidly solidified solids;

(b) solidifying the plurality of rapidly solidified solids to form a solidified molding, wherein:

the magnesium alloy comprises x at % of Zn, y at % of at least one element of Ca and Sr, and the balance of Mg and inevitable impurities; and

x and y satisfy formulae 1 and 2 below.

0.15≤x≤1.5 (Formula 1)

0.5≤y≤1.5 (Formula 2)

[11] The method of manufacturing an implant magnesium alloy according to item [10], further comprising, after the step (b), a step of extruding the solidified molding.

[12] The method of manufacturing an implant magnesium alloy according to item [10] or [11], wherein the magnesium alloy comprises a at % of Mn, and a satisfies formula 3 below.

0.01≤a≤0.08 (Formula 3)

[13] The method of manufacturing an implant magnesium alloy according to any one of items [10] to [12], wherein the magnesium alloy comprises z at % of RE (rare-earth element), and z satisfies formula 4 below.

0≤z≤0.2 (Formula 4)

[14] The method of manufacturing an implant magnesium alloy according to any one of items [10] to [13], wherein:

the rapidly solidified solid or the solidified molding has a plurality of a-Mg grains; and

the plurality of a-Mg grains have an average grain diameter of 0.8 μm or more and 2.5 μm or less.

[15] The method of manufacturing an implant magnesium alloy according to any one of items [10] to [14], wherein the rapidly solidified solid or the solidified molding has at least one compound of Mg₂Ca and Mg₁₇Sr₂.

[16] The method of manufacturing an implant magnesium alloy according to item [15], wherein the at least one compound has an average grain diameter of 0.07 μm or more and 0.29 μm or less.

[17] The method of manufacturing an implant magnesium alloy according to any one of items [10] to [16], wherein the rapidly solidified solid or the solidified molding does not have a compound of Mg₆Ca₂Zn₃ in a constituent phase determined by X-ray diffraction.

[18] The method of manufacturing an implant magnesium alloy according to any one of items [10] to [17], wherein the magnesium alloy does not contain an unavoidable amount or more of Al.

[19] A method of manufacturing a bone fixture, comprising manufacturing a bone fixture by using the implant magnesium alloy according to any one of items [10] to [18].

According to one aspect of the present invention, an implant magnesium alloy having both corrosion resistance and mechanical strength, a bone fixture, a method of manufacturing an implant magnesium alloy, or a method of manufacturing a bone fixture can be provided.

### BRIEF DESCRIPTION OF DRAWINGS

FIG. 1 is a schematic diagram of an apparatus system for manufacturing rapidly solidified magnesium alloy flakes by using single-roller melt spinning;

FIG. 2 is a diagram illustrating Zn additive amount dependence of yield strength σ_{YS} and elongation δ in rapidly solidified ribbon-consolidated Mg₉₉₋ₓCa₁Znₓ alloys in Examples 1 to 3 and Comparative Examples 1 to 3;

FIG. 3 is a diagram illustrating Zn additive amount dependence of corrosion rates of the rapidly solidified ribbon-consolidated Mg₉₉₋ₓCa₁Znₓ alloys in Examples 1 to 3 and Comparative Examples 1 to 3 in simulated body fluid;

FIG. 4 is a diagram illustrating an X-ray diffraction chart of the rapidly solidified ribbon-consolidated Mg₉₉₋ₓCa₁Znₓ alloys (x=0, x=0.2, x=1, x=2, x=3) in Examples 1 and 2 and Comparative Examples 1 to 3;

FIG. 5 is a diagram illustrating extrusion temperature dependence of yield strength σ_{YS} and elongation δ in rapidly solidified ribbon-consolidated Mg_{98.5}Ca₁Zn_{0.5} alloys in Examples 3 to 7 during consolidation;

FIG. 6 is a diagram illustrating a nominal stress-strain curve of rapidly solidified ribbon-consolidated Mg_{98.5-a}Ca₁Zn_{0.5}Mnₐ (a=0, 0.03 at %) in each of Example 3 and Example 8;

FIG. 7 is a bar chart illustrating the corrosion rate of rapidly solidified ribbon-consolidated Mg_{98.5-a}Ca₁Zn_{0.5}Mnₐ (a=0, 0.03 at %) in each of Example 3 and Example 8 and a WE43 cast-extruded material in Comparative Example 7 in simulated body fluid;

FIG. 8 is a tissue diagram illustrating grain-size distribution and crystal orientation obtained by measuring the rapidly solidified ribbon-consolidated Mg_{98.5}Ca₁Zn_{0.5} alloy in Example 3 by EBSD;

FIG. 9 is an SEM photograph illustrating material tissue of each of the rapidly solidified ribbon-consolidated Mg_{98.5}Ca₁Zn_{0.5} alloy in Example 3, a rapidly solidified ribbon-consolidated Mg_{98.5}Ca_{0.5}Sr_{0.5}Zn_{0.5} alloy in Example 9, and a rapidly solidified ribbon-consolidated Mg_{98.5}Sr₁Zn_{0.5} alloy in Example 10;

FIG. 10 is a diagram illustrating an X-ray diffraction chart of each of the rapidly solidified ribbon-consolidated Mg_{98.5}Ca₁Zn_{0.5} alloy in Example 3, the rapidly solidified ribbon-consolidated Mg_{98.5}Ca_{0.5}Sr_{0.5}Zn_{0.5} alloy in Example 9, and the rapidly solidified ribbon-consolidated Mg_{98.5}Sr₁Zn_{0.5} alloy in Example 10;

FIG. 11 is a diagram illustrating yield strength σ_{YS} and elongation δ in each of the alloys in Examples 3, 8, and 10 and alloys in Examples 12 and 13 and Comparative Example 8 obtained by adding Y to the respective alloys;

FIG. 12 is a bar chart illustrating the corrosion rate of each of the alloys in Examples 3, 8, and 10 and the alloys in Examples 12 and 13 and Comparative Example 8 obtained by adding Y to the respective alloys in simulated body fluid;

FIG. 13 is a diagram illustrating an X-ray diffraction chart (XRD) of each of cast-extruded materials in Comparative Examples 4, 5, and 6; and

FIG. 14 is an SEM photograph of the cross section of a Mg_{98.5}Ca₁Zn_{0.5} cast-extruded material (extrusion temperature: 350° C.) in Comparative Example 5.

### DESCRIPTION OF EMBODIMENTS

Embodiments of the present invention are described in detail below with reference to the drawings. The present invention is not limited to the following description, and a person skilled in the art could easily understand that the forms and details of the present invention can be variously changed without departing from the gist and scope of the present invention. Thus, the present invention is not intended to be interpreted as being limited to the following description of the embodiments.

A bone fixture according to one aspect of the present invention is described. Examples of the bone fixture include a plate, a screw, a clip, and a bolt used for therapy for bone fracture. The bone fixture is preferably formed from an implant magnesium alloy having a property of being absorbed to a biological body and having biological affinity. The reason is that the implant magnesium alloy is harmless even when absorbed to a biological body during therapy or after therapy is finished. The implant magnesium alloy does not contain Al or Ni, which is harmful to biological bodies, but contains Ca that promotes the regeneration of bone tissue.

An implant magnesium alloy according to one aspect of the present invention contains x at % of Zn, a total of y at % of at least one element of Ca and Sr, and the balance of Mg and inevitable impurities. x and y may satisfy formulae 1 and 2 below.

0.15≤x≤1.5 (preferably 0.2≤x≤1.0) (Formula 1)

0.5≤y≤1.5 (preferably 0.75≤y≤1.25) (Formula 2)

Note that an implant is a generic term for a device to be embedded in the body.

The reason why the content range of Zn should satisfy Formula 1 above is that when the content of Zn exceeds 1.5 at %, a compound (for example, Mg₆Ca₂Zn₃ compound) other than Mg₂Ca and Mg₁₇Sr₂ is produced so that corrosion resistance decreases and the corrosion rate in simulated body fluid becomes higher than 0.7 mm/year, and even when the content of Zn is less than 0.15 at %, corrosion resistance decreases so that the corrosion rate in simulated body fluid becomes higher than 0.7 mm/year.

The case where the alloy contains a total of y at % of at least one element of Ca and Sr as described above includes a case where the alloy contains 0.5 at % or more and 1.5 at % or less of Ca, a case where the alloy contains a total of 0.5 at % or more and 1.5 at % or less of Ca and Sr, and a case where the alloy contains 0.5 at % or more and 1.5 at % or less of Sr. Ca has an effect of promoting the regeneration of bone tissue, but even when Ca is replaced with Sr, the same effect is considered to be provided.

The reason why the content range of Ca should satisfy Formula 2 above is that when the total content of Ca and Sr exceeds 1.5 at %, ductility and corrosion resistance decrease, and when the total content of Ca and Sr is less than 0.5 at %, an effect of promoting the regeneration of bone tissue cannot be obtained, and corrosion resistance and mechanical strength decrease.

The above-mentioned implant magnesium alloy may further contain a at % of Mn, and a may satisfy formula 3 below. Containing Mn can decrease the corrosion rate in the body.

0.01≤a≤0.08 (preferably 0.02≤a≤0.05) (Formula 3)

The reason why the content range of Mn should satisfy Formula 3 above is that when the content of Mn exceeds 0.08 at %, ductility decreases, and when the content of Mn is less than 0.01 at %, sufficient corrosion resistance is not obtained.

The above-mentioned implant magnesium alloy may further contain z at % of RE (rare-earth element), and z may satisfy formula 4 below.

0≤z≤0.2 (preferably 0≤z≤0.1) (Formula 4)

RE (rare-earth elements) are Y, La, Ce, Pr, Nd, Pm, Sm, Eu, Gd, Tb, Dy, Ho, Er, Tm, Yb, and Lu.

The reason why the content range of RE should satisfy Formula 4 above is that when RE is contained, ignition temperature of the alloy improves, and when the content of RE exceeds 0.2 at %, ductility and corrosion resistance decrease.

The above-mentioned implant magnesium alloy may have a plurality of a-Mg grains, and the plurality of a-Mg grains may have an average grain diameter of 0.8 μm or more and 2.5 μm or less (preferably 1.0 μm or more and 2.0 μm or less).

When the average grain diameter of the plurality of a-Mg grains exceeds 2.5 μm, mechanical strength and corrosion resistance decrease. When the average grain diameter of the plurality of a-Mg grains is less than 0.8 μm, ductility decreases.

The above-mentioned implant magnesium alloy may have at least one compound of Mg₂Ca and Mg₁₇Sr₂, and the at least one compound may have an average grain diameter of 0.07 μm or more and 0.29 μm or less (preferably 0.09 μm or more and 0.25 μm or less).

When the average grain diameter of the at least one compound exceeds 0.29 μm, corrosion resistance and mechanical strength decrease. When the average grain diameter of the plurality of a-Mg grains is less than 0.07 μm, ductility decreases.

Next, a method of manufacturing an implant magnesium alloy according to one aspect of the present invention is described.

First, a magnesium alloy having the above-mentioned composition is melted at high temperature to manufacture an alloy molten metal, and the molten metal is rapidly solidified at a cooling rate of 1×10³ K/sec or more and 1×10⁶ K/sec or less (preferably 1×10⁴ K/sec or more) to manufacture a plurality of rapidly solidified solids, thereby obtaining powder, flakes, ribbons, or wires as rapidly solidified solids thereof. The plurality of obtained rapidly solidified solids are preformed, and after that, the resultant solidified molding is subjected to plastic working to manufacture a consolidated solidified molding. The preforming may include a step of billet forming by compressing powder, flakes, ribbons, or wires, or may be canning. The preforming has a purpose of facilitating consolidation, and has effects of preventing oxidation during consolidation by solidifying powder and of facilitating handling.

In the plastic working, hot extrusion can be used. For example, hot extrusion is preferably performed under conditions of an extrusion temperature of 275 to 425° C. (preferably 300° C. to 375° C.), an extrusion pressure of 200 to 1,000 MPa, an extrusion ratio of 5 to 100 (preferably 10 to 50), and a ram speed of 0.05 mm/sec or more. Examples of rapid solidification include a gun method, a piston-anvil method, a centrifugation method, a single-roller method, a double-roller method, a spray method, a high-pressure gas jetting method, an in-rotating liquid spinning method, and a thin molten metal injection molding method. The single-roller method or the high-pressure gas jetting method is particularly suitable.

A specific example where a magnesium alloy is rapidly solidified and preformed and the resultant solidified molding is subjected to hot extrusion to be solidified is described below.

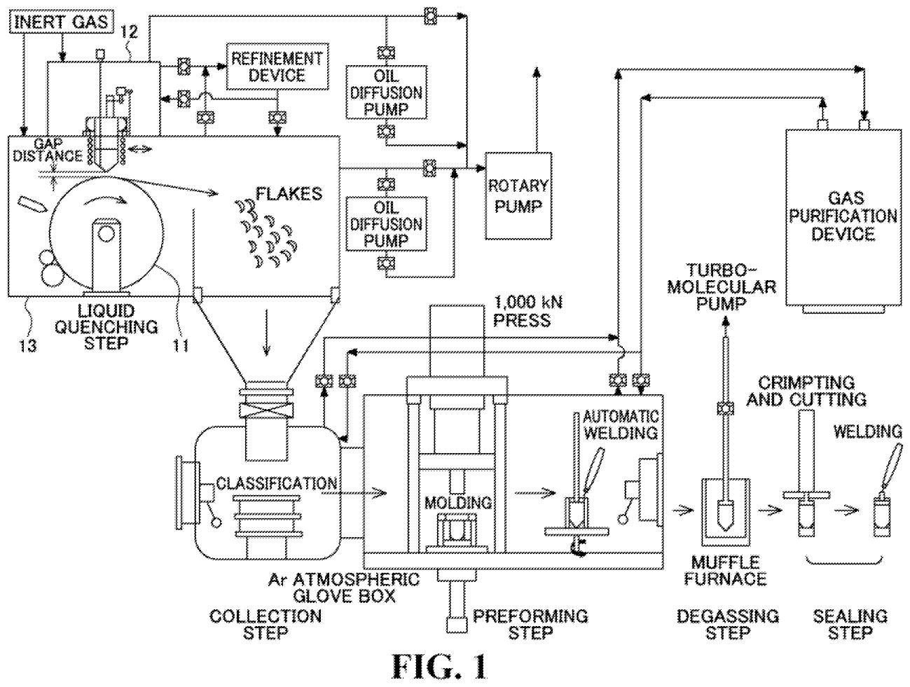

FIG. 1 is a schematic diagram of an apparatus system for manufacturing rapidly solidified magnesium alloy flakes by using single-roller melt spinning.

In a melt spinning step using a single roller 11, first, the above-mentioned implant magnesium alloy is melted by high-frequency induction heating. In this case, in order to prevent segregation of molten metal components, the molten metal is agitated for homogenization. The molten metal is jetted in a manner that inert gas is introduced into a melting chamber 12 and a roller chamber 13 and its differential pressure is controlled. Nozzles and a crucible are integrated and can be moved in the up-down and front-back directions, and the tilt angle of jetting can be changed, so that the form can be controlled such as triangular flakes, foils, and ribbons, thereby manufacturing rapidly solidified magnesium alloy flakes that are optimum for a powder metallurgy process. The up-down direction is a gap distance between the roller 11 and the nozzle distal end, and the front-back direction is a front-back distance from a roller core. Each movement range is 0 to 50 mm. The maximum circumferential speed of the roller is 52 m/sec, and rapidly solidified fine tissue is implemented by high cooling performance in combination with water cooling. The cooling rate is 1×10⁵ K/sec or more.

The produced rapidly solidified magnesium alloy flake has a plurality of a-Mg grains. The average grain diameter of the plurality of a-Mg grains at this time is 10 μm or less. Note that the average grain diameter as used herein is an average value of grain diameters measured by using SEM and TEM.

The mass-produced rapidly solidified magnesium alloy flakes are filtered and classified in an argon atmosphere glove box in a collection step, and are thereafter loaded in a capsule made of copper or aluminum. Preforming is performed by a press with an output of 1,000 kN, and a lid portion with a deaeration tube is welded to the capsule (preforming step). A valve is attached to the deaeration tube, and the capsule is taken out while the argon gas atmosphere inside the capsule is maintained. Vacuum degassing is performed by a turbo-molecular pump while heating the capsule to a predetermined temperature (degassing step). After degassing, the deaeration tube is crimped and cut/welded to manufacture a billet (sealing step). Note that the argon atmosphere glove box is directly coupled to a gas purification device, and hence by interrupting the connection to a single-roller melt spinning device through a gate valve, the rapidly solidified magnesium alloy flakes are not exposed to air at all until a billet is manufactured.

After that, the billet is consolidated by extrusion at an extrusion temperature of 275° C. to 425° C., a ram speed of 0.05 mm/sec or more, and an extrusion ratio of 5 to 100. Through the extrusion, pressure and shear are applied to the magnesium alloy flakes, so that densification and bonding between flakes are achieved. Note that shear occurs also in forming by rolling or casting.

The rapidly solidified ribbon solidified molding (implant magnesium alloy) obtained by the consolidation has a plurality of a-Mg grains, and has a fine crystal tissue in which the average grain diameter of the a-Mg grains is 0.8 μm or more and 2.5 μm or less (preferably 1.0 μm or more and 2.0 μm or less). The consolidated solidified molding has at least one compound of Mg₂Ca and Mg₁₇Sr₂, and the average grain diameter of the at least one compound is 0.07 μm or more and 0.29 μm or less (preferably 0.09 μm or more and 0.25 μm or less).

The rapidly solidified ribbon solidified molding manufactured by rapid solidification as described above has a fine isotropic tissue, and hence the anisotropy of strengths in the rapidly solidified ribbon solidified molding can be reduced.

A workpiece may be manufactured by extrusion, rolling, or casting of the above-mentioned rapidly solidified ribbon solidified molding. By using the rapidly solidified ribbon solidified molding or its workpiece, a step of manufacturing a bone fixture can be implemented.

Note that, if necessary, heat treatment may be performed before or after the step of manufacturing a rapidly solidified solid, the step of manufacturing a solidified molding, the step of manufacturing a workpiece, and the step of manufacturing a bone fixture.

The above-mentioned implant magnesium alloy can also be used for bioabsorbable medical devices other than the above-mentioned bone fixture.

According to the present embodiment, a molten metal of a magnesium alloy is rapidly solidified to manufacture a rapidly solidified solid, and an implant magnesium alloy is manufactured by the rapidly solidified solid. Thus, the average grain diameter of a-Mg grains can be reduced to 2.5 μm or less (preferably 2.0 μm or less). As a result, both corrosion resistance and mechanical strength/ductility of the implant magnesium alloy can be enhanced. In other words, when a bone fixture using the implant magnesium alloy is embedded in the body, the bioresorption rate can be reduced, and corrosion resistance sufficient for use as a bone fixture can be achieved, and further necessary strength as a bone fixture can be achieved. For example, sufficient corrosion resistance means that the corrosion rate in simulated body fluid is 0.01 to 0.7 mm/year (preferably 0.01 to 0.55 mm/year), and necessary strength means that tensile yield strength (yield strength) is 260 MPa or more (preferably 280 MPa or more, more preferably 300 MPa or more) and elongation is 7% or more (preferably 12% or more).

In the present embodiment, a molten metal of a magnesium alloy is rapidly solidified to manufacture a rapidly solidified solid, and an implant magnesium alloy is manufactured by the rapidly solidified solid, and hence the implant magnesium alloy has a fine homogeneous tissue. Thus, a micro part for implants with stable quality can be manufactured.

In the present embodiment, the implant magnesium alloy has at least one compound of Mg₂Ca and Mg₁₇Sr₂, and the at least one compound has an average grain diameter of 0.07 μm or more and 0.29 μm or less (preferably 0.09 μm or more and 0.25 μm or less). Because the implant magnesium alloy has such a compound, coarsening of crystal grains can be suppressed in steps of manufacturing an implant magnesium alloy and a bone fixture using the implant magnesium alloy. As a result, the decrease in strength, ductility, and corrosion resistance of the alloy can be suppressed.

The implant magnesium alloy according to the present embodiment has high strength, high ductility, and high corrosion resistance as compared to WE43, which is an existing bioabsorbable magnesium alloy. Note that WE43 is a code for ASTM (USA), and is a magnesium alloy including 4 wt % of Y and 3 wt % of rare-earth element.

The implant magnesium alloy according to the present embodiment contains a total of 0.5 at % or more of Ca and Sr, and hence when the implant magnesium alloy is used as a bone fixture, the regeneration of bone tissue can be promoted.

Note that, in the present embodiment, the implant magnesium alloy in which the average grain diameter of a-Mg grains is small and the average grain diameter of compounds of Mg₂Ca and Mg₁₇Sr₂ is small is manufactured by a rapid solidification method. However, an implant magnesium alloy in which the average grain diameter of a-Mg grains and the average grain diameter of the compounds are small may be manufactured by a severe plastic deformation method, where severe plastic deformation is applied to a cast material. Examples of the severe plastic deformation method include equal channel angular extrusion (ECAE), high-pressure torsion (HPT), accumulative roll bonding (ARB), multi directional forging (MDF), and high-pressure sliding (HPS).

The ECAE is a method in which, in order to introduce uniform distortion to a sample, a sample longitudinal direction is rotated by 90° for each pass. Specifically, a magnesium alloy cast as a forming material is forcedly caused to enter a molding hole having an L-shaped cross section in a molding die, and stress is applied to the magnesium alloy cast particularly at a 90°-bent portion of the L-shaped molding hole to obtain a solidified molding. The number of passes in ECAE is preferably multiple. Temperature in ECAE is preferably, for example, 275° C. or more and 425° C. or less. Note that the above-mentioned magnesium alloy cast is obtained by melting and casting a magnesium alloy having the above-mentioned composition.

By the above-mentioned severe plastic deformation method, a-Mg grains can be made fine to reduce the average grain diameter, and the compounds of Mg₂Ca and Mg₁₇Sr₂ can be ground and dispersed to reduce the average grain diameter.

### EXAMPLES

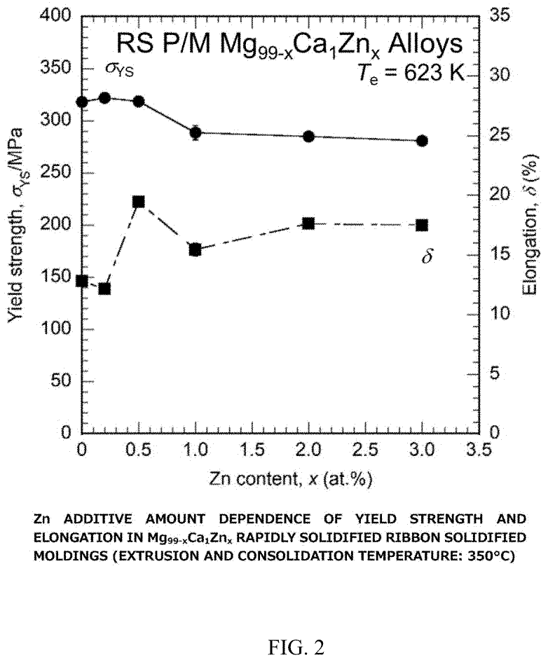

FIG. 2 is a diagram illustrating Zn additive amount dependence of tensile yield strength and elongation in rapidly solidified ribbon solidified moldings (extrusion and consolidation temperature: 350° C.) of Mg₉₉₋ₓCa₁Znₓ alloys in Examples 1 to 3 and Comparative Examples 1 to 3 as implant magnesium alloys. In Example 1, x is 0.2. In Example 2, x is 1. In Example 3, x is 0.5. In Comparative Example 1, x is 0. In Comparative Example 2, x is 2. In Comparative Example 3, x is 3.

The Mg₉₉₋ₓCa₁Znₓ alloys in Examples 1 to 3 and Comparative Examples 1 to 3 were manufactured by rapidly solidified ribbon-consolidated formation. A specific manufacturing method is as follows.

An alloy having a composition of Mg₉₉₋ₓCa₁Znₓ is melted by high-frequency heating under argon gas atmosphere, and is cooled at a cooling rate of about 2×10⁵ K/sec by using a single-roller melt spinning device to manufacture a quenched ribbon. Next, the quenched ribbon is preformed at a pressure of 60 to 170 MPa, and subjected to vacuum degassing for 2 hours at a temperature of 250° C. to manufacture a billet. Consolidation is performed by extrusion at an extrusion temperature of 350° C., a ram speed of 0.05 mm/sec or more, and an extrusion ratio of 10 or more to manufacture a rapidly solidified ribbon solidified molding, thereby manufacturing a Mg₉₉₋ₓCa₁Znₓ alloy in each of Examples 1 to 3 and Comparative Examples 1 to 3.

Next, tensile testing was performed on the Mg₉₉₋ₓCa₁Znₓ alloys in Examples 1 to 3 and Comparative Examples 1 to 3 under room temperature. The results are shown in FIG. 2 and Table 1. The horizontal axis in FIG. 2 indicates the content of Zn, the left vertical axis indicates tensile yield strength (Sys, and the right vertical axis indicates elongation δ. According to FIG. 2 and Table 1, Examples 1 to 3 exhibited a tensile yield strength (yield strength) of 280 MPa or more and an elongation of 12% or more, thereby achieving both the tensile yield strength and the elongation.

TABLE 1

| | | | | | | Extrusion and |
|---|---|---|---|---|---|---|
| | | | | Break- | Corrosion | consolidation |
| | | | Tensile | ing | speed in | temperature or |
| | | | yield | elonga- | simulated | cast-extrusion |
| | Manufacturing | Alloy | strength | tion | body fluid | temperature |
| | method | (at %) | (MPa) | (%) | (mm/year) | (K) |
| Comparative | Rapidly solidified | Mg₉₉Ca₁ | 318 | 12.8 | >1.01 | 623 |
| Example 1 | ribbon consolidation | | | | | |
| Comparative | Rapidly solidified | Mg₉₇Ca₁Zn₂ | 285 | 17.6 | 0.85 | 623 |
| Example 2 | ribbon consolidation | | | | | |
| Comparative | Rapidly solidified | Mg₉₆Ca₁Zn₃ | 281 | 17.5 | >1.00 | 623 |
| Example 3 | ribbon consolidation | | | | | |
| Comparative | Cast-extrusion | Mg_{95.8}Ca₁Zn_{0.2} | 197 | 15.2 | >1.00 | 623 |
| Example 4 | | | | | | |
| Comparative | Cast-extrusion | Mg₉₈Ca₁Zn₁ | 215 | 15.9 | >1.00 | 623 |
| Example 5 | | | | | | |
| Comparative | Cast-extrusion | Mg₉₈Ca₁Zn₁ | 217 | 17.5 | >1.00 | 623 |
| Example 6 | | | | | | |
| Comparative | Cast-extrusion | WE43 | 210 | 12.0 | 0.52 | 623 |
| Example 7 | | | | | | |
| Comparative | Rapidly solidified | Mg_{98.4}Sr₁Zn_{0.5}Y_{0.1} | 455 | 5.1 | 0.08 | 623 |
| Example 8 | ribbon consolidation | | | | | |
| Example 1 | Rapidly solidified | Mg_{98.8}Ca₁Zn_{0.2} | 322 | 12.2 | 0.54 | 623 |
| | ribbon consolidation | | | | | |
| Example 2 | Rapidly solidified | Mg₉₈Ca₁Zn₁ | 289 | 15.5 | 0.53 | 623 |
| | ribbon consolidation | | | | | |
| Example 3 | Rapidly solidified | Mg_{98.5}Ca₁Zn_{0.5} | 319 | 19.5 | 0.27 | 623 |
| | ribbon consolidation | | | | | |
| Example 4 | Rapidly solidified | Mg_{98.5}Ca₁Zn_{0.5} | 414 | 7.5 | 0.25 | 573 |
| | ribbon consolidation | | | | | |
| Example 5 | Rapidly solidified | Mg_{98.5}Ca₁Zn_{0.5} | 371 | 13.3 | 0.28 | 598 |
| | ribbon consolidation | | | | | |
| Example 6 | Rapidly solidified | Mg_{98.5}Ca₁Zn_{0.5} | 326 | 17.5 | 0.27 | 608 |
| | ribbon consolidation | | | | | |
| Example 7 | Rapidly solidified | Mg_{98.5}Ca₁Zn_{0.5} | 267 | 20.6 | 0.29 | 648 |
| | ribbon consolidation | | | | | |
| Example 8 | Rapidly solidified | Mg_{98.47}Ca₁Zn_{0.5}Mn_{0.03} | 322 | 19.0 | 0.08 | 623 |
| | ribbon consolidation | | | | | |
| Example 9 | Rapidly solidified | Mg_{98.5}Ca_{0.5}Sr_{0.5}Zn_{0.5} | 375 | 12.7 | 0.32 | 623 |
| | ribbon consolidation | | | | | |
| Example 10 | Rapidly solidified | Mg_{98.5}Sr₁Zn_{0.5} | 403 | 12.7 | 0.38 | 623 |
| | ribbon consolidation | | | | | |
| Example 11 | Severe plastic | Mg_{98.5}Ca₁Zn_{0.5} | 312 | 13.2 | 0.39 | 623 |
| | deformation of cast | | | | | |
| Example 12 | Rapidly solidified | Mg_{98.5}Ca₁Zn_{0.5}Y_{0.1} | 356 | 17.0 | 0.19 | 623 |
| | ribbon consolidation | | | | | |
| Example 13 | Rapidly solidified | Mg_{98.37}Ca₁Zn_{0.5}Mn_{0.03}Y_{0.1} | 378 | 15.3 | 0.11 | 623 |
| | ribbon consolidation | | | | | |
| Example 14 | Rapidly solidified | Mg_{98.4}Sr₁Zn_{0.5}Y_{0.1} | 407 | 8.3 | 0.09 | 648 |
| | ribbon consolidation | | | | | |

| | α-Mg | Compound | |
|---|---|---|---|
| | grain | grain | |
| | diameter | diameter | |
| | (μm) | (μm) | Constituent phase determined by XRD |
| Comparative | 1.4 | 0.13 | α-Mg + Mg₂Ca |
| Example 1 | | | |
| Comparative | 1.5 | 0.15 | α-Mg + Mg₂Ca + Mg₆Ca₂Zn₃ |
| Example 2 | | | |
| Comparative | 1.6 | 0.18 | α-Mg + Mg₂Ca + Mg₆Ca₂Zn₃ |
| Example 3 | | | |
| Comparative | 2.6 | 0.24 | α-Mg + Mg₂Ca + Mg₆Ca₂Zn₃ |
| Example 4 | | | |
| Comparative | 2.9 | 0.32 | α-Mg + Mg₂Ca + Mg₆Ca₂Zn₃+ Mg₄Zn₇ |
| Example 5 | | | |
| Comparative | 3.5 | 0.48 | α-Mg + Mg₂Ca + Mg₆Ca₂Zn₃+ Mg₄Zn₇ |
| Example 6 | | | |
| Comparative | 2.7 | 0.30 | — |
| Example 7 | | | |
| Comparative | 0.9 | 0.30 | α-Mg + Mg₁₇Sr₂ |
| Example 8 | | | |
| Example 1 | 1.5 | 0.12 | α-Mg + Mg₂Ca |
| Example 2 | 1.5 | 0.13 | α-Mg + Mg₂Ca |
| Example 3 | 1.4 | 0.13 | α-Mg + Mg₂Ca |
| Example 4 | 1.1 | 0.09 | α-Mg + Mg₂Ca |
| Example 5 | 1.1 | 0.10 | α-Mg + Mg₂Ca |
| Example 6 | 1.5 | 0.12 | α-Mg + Mg₂Ca |
| Example 7 | 1.9 | 0.13 | α-Mg + Mg₂Ca |
| Example 8 | 1.3 | 0.13 | α-Mg + Mg₂Ca |
| Example 9 | 1.0 | 0.11 | α-Mg + Mg₂Ca + Mg₁₇Sr₂ |
| Example 10 | 1.1 | 0.11 | α-Mg + Mg₁₇Sr₂ |
| Example 11 | 1.4 | 0.12 | α-Mg + Mg₂Ca |
| Example 12 | 1.3 | 0.23 | α-Mg + Mg₂Ca |
| Example 13 | 1.1 | 0.17 | α-Mg + Mg₂Ca |
| Example 14 | 1.2 | 0.22 | α-Mg + Mg₁₇Ca |

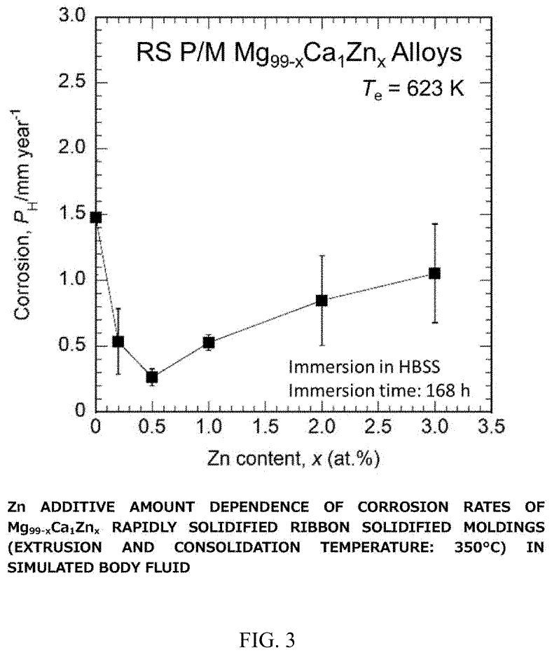

FIG. 3 is a diagram illustrating Zn additive amount dependence of corrosion rates of the rapidly solidified ribbon solidified moldings (extrusion and consolidation temperature: 350° C.) of the Mg₉₉₋ₓCa₁Znₓ alloys in Examples 1 to 3 and Comparative Examples 1 to 3 in simulated body fluid.

The corrosion rate was measured by immersing each of the alloys in Examples 1 to 3 and Comparative Examples 1 to 3 with simulated body fluid (HBSS: physiological balanced salt solution) adjusted to pH 7.4 for 168 hours. The simulated body fluid in the measurement was opened to air at a temperature of 37° C. The measurement results are shown in FIG. 3 and Table 1.

As illustrated in FIG. 3, it was confirmed that the alloys in Examples 1 to 3 had corrosion resistance higher than that of the alloys in Comparative Examples 1 to 3.

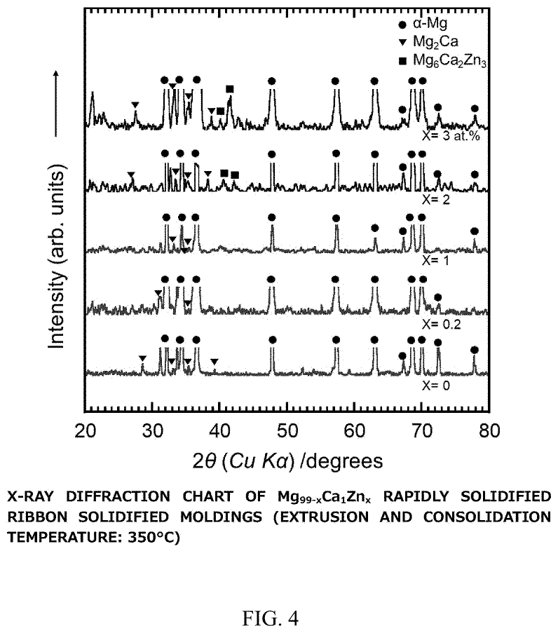

FIG. 4 is a diagram illustrating an X-ray diffraction chart of the rapidly solidified ribbon solidified moldings (extrusion and consolidation temperature: 350° C.) of the Mg₉₉₋ₓCa₁Znₓ alloys (x=0, x=0.2, x=1, x=2, x=3) in Examples 1 and 2 and Comparative Examples 1 to 3.

As illustrated in FIG. 4, when the additive amount of Zn is 2 at % or more (Comparative Example 2 and Comparative Example 3), a Mg₆Ca₂Zn₃ compound is produced. Thus, in Comparative Examples 2 and 3, a compound (for example, Mg₆Ca₂Zn₃ compound) other than Mg₂Ca and Mg₁₇Sr₂ is produced, and the corrosion resistance decreases as illustrated in FIG. 3. Accordingly, it is preferred that no Mg₆Ca₂Zn₃ compound be contained in a magnesium alloy.

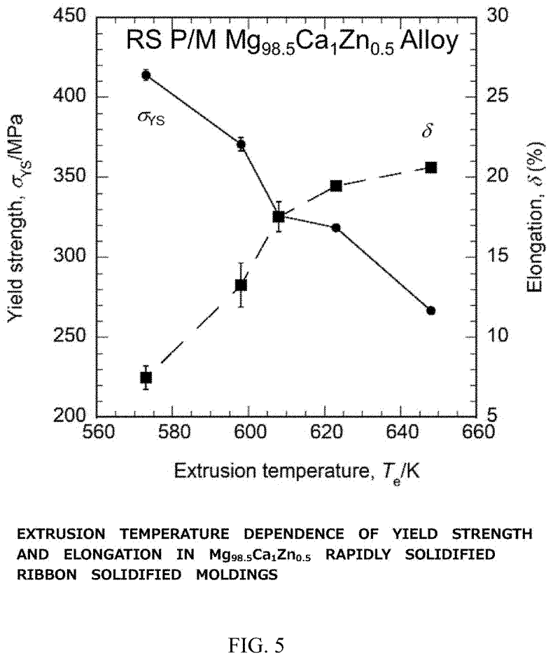

FIG. 5 is a diagram illustrating extrusion and consolidation temperature dependence of tensile yield strength and elongation in rapidly solidified ribbon solidified moldings of Mg_{98.5}Ca₁Zn_{0.5} alloys in Examples 3 to 7 as implant magnesium alloys during consolidation.

The Mg_{98.5}Ca₁Zn_{0.5} alloys in Examples 4 to 7 were manufactured by the same rapidly solidified ribbon consolidation method as the Mg₉₉₋ₓCa₁Znₓ alloys in Examples 1 to 3 except for the extrusion temperature for consolidation. The extrusion and consolidation temperature during consolidation in Example 3 is 350° C., but the extrusion and consolidation temperature during consolidation in Example 4 is 300° C., the extrusion and consolidation temperature during consolidation in Example 5 is 325° C., the extrusion and consolidation temperature during consolidation in Example 6 is 335° C., and the extrusion and consolidation temperature during consolidation in Example 7 is 375° C.

Next, tensile testing was performed on each of the Mg_{98.5}Ca₁Zn_{0.5} alloys in Examples 4 to 7 under room temperature. The results are shown in FIG. 5 and Table 1. The horizontal axis in FIG. 5 indicates extrusion and consolidation temperature, the left vertical axis indicates tensile yield strength 6 μs, and the right vertical axis indicates elongation δ. According to FIG. 5 and Table 1, Examples 4 to 7 exhibited a tensile yield strength (yield strength) of 260 MPa or more and an elongation of 7% or more, thereby achieving both the tensile yield strength and the elongation.

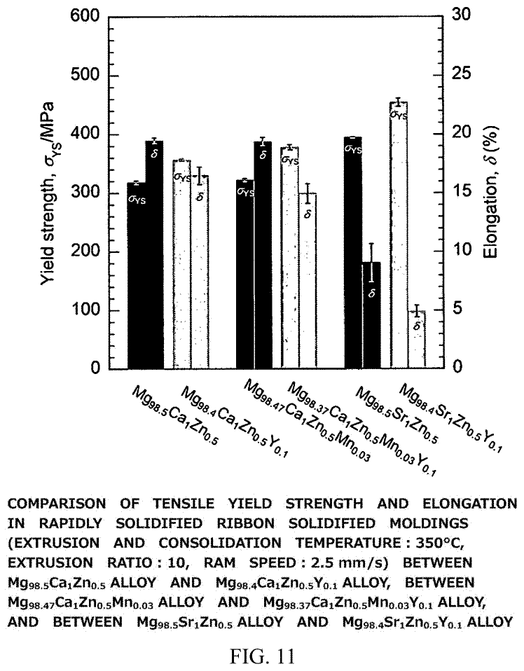

FIG. 11 is a diagram illustrating the influence of Y addition on mechanical properties of quenched alloys, and is a diagram illustrating the comparisons of tensile yield strength and elongation in rapidly solidified ribbon solidified moldings (extrusion and consolidation temperature: 350° C., extrusion ratio: 10, ram speed: 2.5 mm/s) between the Mg_{98.5}Ca₁Zn_{0.5} alloy in Example 3 and the Mg_{98.4}Ca₁Zn_{0.5}Y_{0.1} alloy in Example 12, between the Mg_{98.47}Ca₁Zn_{0.5}Mn_{0.03} alloy in Example 8 and the Mg_{98.37}Ca₁Zn_{0.5}Mn_{0.03}Y_{0.1} alloy in Example 13, and between the Mg_{98.5}Sr₁Zn_{0.5} alloy in Example 10 and the Mg_{98.4}Sr₁Zn_{0.5}Y_{0.1} alloy in Comparative Example 8 as implant magnesium alloys. Those magnesium alloys were manufactured by the same rapidly solidified ribbon consolidation method as in Examples 1 to 3.

As illustrated in FIG. 11, the yield strength σ_{YS} was able to be improved by adding Y.

Table 1 shows the experimental results in Example 3, Example 8, Example 10, Example 12, and Example 13. Example 12 (Mg_{98.4}Ca₁Zn_{0.5}Y_{0.1} alloy) and Example 13 (Mg_{98.37}Ca₁Zn_{0.5}Mn_{0.03}Y_{0.1} alloy) exhibit an elongation of 7% or more.

In Comparative Example 8 (Mg_{98.4}Sr₁Zn_{0.5}Y_{0.1} alloy) above, the elongation was 5% lower than 7%. In Comparative Example 8, when the extrusion and consolidation temperature is 350° C., the alloy exhibits an extremely high yield strength, but the elongation is insufficient. However, as shown in Table 1, it was found that in Example 14, when the extrusion and consolidation temperature was set to as high as 375° C., the elongation was able to be improved to 8.3% equal to or higher than 7% although the yield strength slightly decreased to 407 MPa. Note that the magnesium alloy in Example 14 was manufactured by the same rapidly solidified ribbon consolidation method as in Examples 1 to 3.

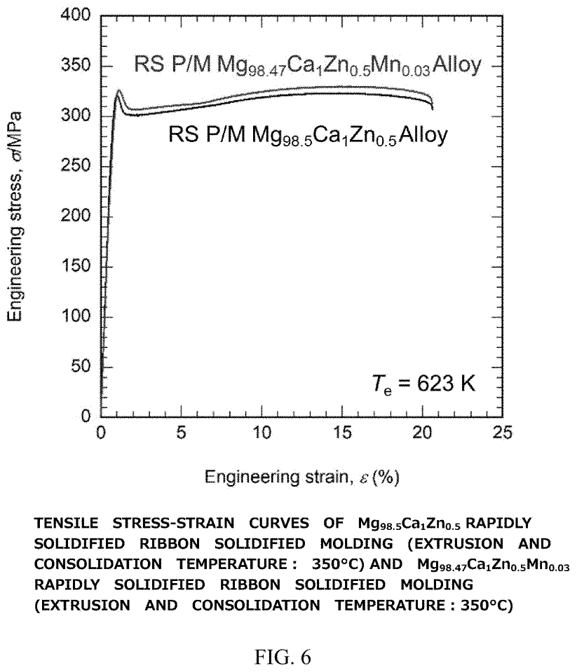

FIG. 6 is a diagram illustrating tensile stress-strain curves of the rapidly solidified ribbon solidified molding (extrusion and consolidation temperature: 350° C.) of the Mg_{98.5}Ca₁Zn_{0.5} alloy in Example 3 and the rapidly solidified ribbon solidified molding (extrusion and consolidation temperature: 350° C.) of the Mg_{98.47}Ca₁Zn_{0.5}Mn_{0.03} alloy in Example 8, and illustrates the influence of the addition of Mn on mechanical properties.

Example 3 is an alloy that does not contain Mn, and its composition is Mg_{98.5}Ca₁Zn_{0.5.}

Example 8 is an alloy that contains Mn, and its composition is Mg_{98.47}Ca₁Zn_{0.5}Mn_{0.03}.

The Mg_{98.47}Ca₁Zn_{0.5}Mn_{0.03} alloy in Example 8 was manufactured by the same rapidly solidified ribbon consolidation method as the Mg₉₉₋ₓCa₁Znₓ alloys in Examples 1 to 3.

Next, tensile testing was performed on the Mg_{98.47}Ca₁Zn_{0.5}Mn_{0.03} alloy in Example 8. As a result, a nominal stress-strain curve illustrated in FIG. 6 was obtained. Examples 3 and 8 both exhibited a tensile yield strength of 300 MPa or more and an elongation of approximately 20%, thereby achieving both the tensile yield strength and the elongation.

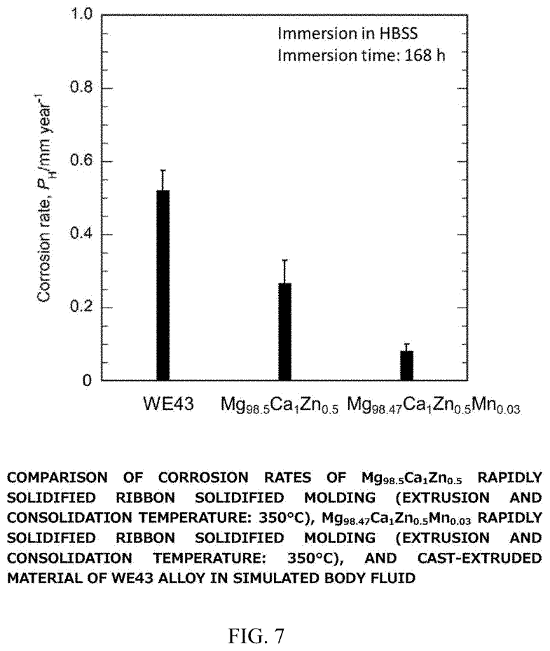

FIG. 7 is a bar chart illustrating the corrosion rate of each of the rapidly solidified ribbon solidified molding (extrusion and consolidation temperature: 350° C.) of the Mg_{98.5}Ca₁Zn_{0.5} alloy in Example 3, the rapidly solidified ribbon solidified molding (extrusion and consolidation temperature: 350° C.) of the Mg_{98.47}Ca₁Zn_{0.5}Mn_{0.03} alloy in Example 8, and a WE43 alloy in Comparative Example 7 in simulated body fluid. The WE43 alloy in Comparative Example 7 is a cast-extruded material.

The corrosion rate measurement method is the same as in Examples 1 to 3.

As illustrated in FIG. 7, it was confirmed that the Mg_{98.5}Ca₁Zn_{0.5} alloy in Example 3 and the Mg₉8.47 Ca₁Zn_{0.5}Mn_{0.03} alloy in Example 8 both had a corrosion resistance higher than that of the WE43 alloy in Comparative Example 7. In particular, it was found that the Mg₉8.47 Ca₁Zn_{0.5}Mn_{0.03} alloy in Example 8 had corrosion resistance that was about five times as high as that of the WE43 alloy in simulated body fluid. Furthermore, it was found from the results in Examples 3 and 8 that the addition of Mn improved the corrosion resistance.

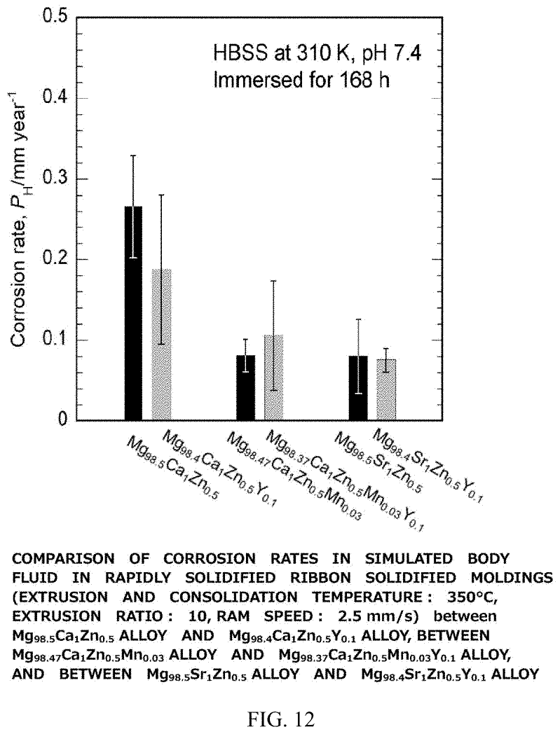

FIG. 12 is a diagram illustrating the influence of Y addition on corrosion rates of quenched alloys in simulated body fluid, and is a bar chart illustrating the comparisons of corrosion rates in simulated body fluid in the rapidly solidified ribbon solidified moldings (extrusion and consolidation temperature: 350° C., extrusion ratio: 10, ram speed: 2.5 mm/s) between the Mg_{98.5}Ca₁Zn_{0.5} alloy in Example 3 and the Mg_{98.4}Ca₁Zn_{0.5}Y_{0.1} alloy in Example 12, between the Mg₉8.47 Ca₁Zn_{0.5}Mn_{0.93} alloy in Example 8 and the Mg_{98.37}Ca₁Zn_{0.5}Mn_{0.03}Y_{0.1} alloy in Example 13, and between the Mg_{98.5}Sr₁Zn_{0.5} alloy in Example 10 and the Mg_{98.4}Sr₁Zn_{0.5}Y_{0.1} alloy in Comparative Example 8.

The corrosion rate measurement method is the same as in Examples 1 to 3.

As illustrated in FIG. 12, it was confirmed that when Y was added to the Mg_{98.5}Ca₁Zn_{0.5} alloy in Example 3, the resultant alloy had a corrosion resistance higher than that of the Mg_{98.5}Ca₁Zn_{0.5} alloy in Example 3. In regard to the other alloys, even the alloy in which Y was not added had high corrosion resistance, and hence even when Y was added, the alloys had the same level of corrosion resistance.

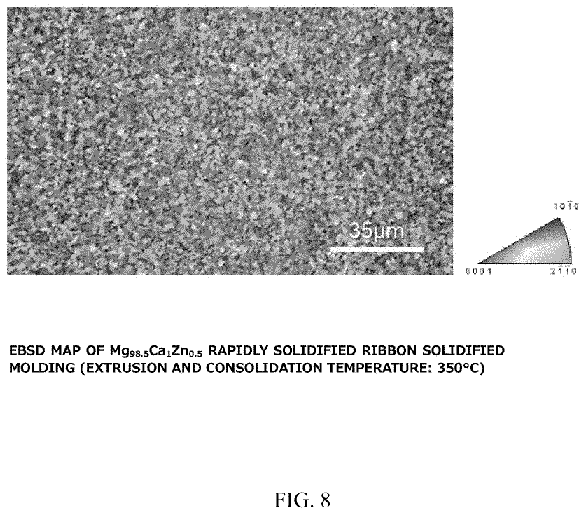

FIG. 8 is a tissue diagram illustrating grain-size distribution and crystal orientation obtained by measuring the Mg_{98.5}Ca₁Zn_{0.5} alloy in Example 3 by electron backscatter diffraction (EBSD).

As illustrated in FIG. 8, it was confirmed that the Mg_{98.5}Ca₁Zn_{0.5} alloy in Example 3 had an isotropic fine tissue. From this, it is considered that the Mg_{98.5}Ca₁Zn_{0.5} alloy in Example 3 has symmetry and isotropy of yield strengths.

The average grain diameter of a-Mg grains in the Mg_{98.5}Ca₁Zn_{0.5} alloy in Example 3 illustrated in FIG. 8 was 1.42 μm.

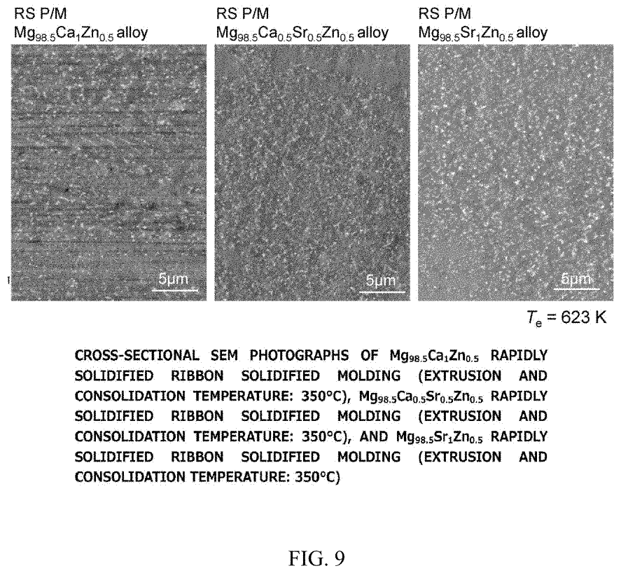

FIG. 9 is an SEM photograph illustrating crystal tissue of the rapidly solidified ribbon solidified molding (extrusion and consolidation temperature: 350° C.) of the Mg_{98.5}Ca₁Zn_{0.5} alloy in Example 3, the rapidly solidified ribbon solidified molding (extrusion and consolidation temperature: 350° C.) of the Mg_{98.5}Ca_{0.5}Sr_{0.5}Zn_{0.5} alloy in Example 9, and the rapidly solidified ribbon solidified molding (extrusion and consolidation temperature: 350° C.) of the Mg_{98.5}Sr₁Zn_{0.5} alloy in Example 10.

The Mg_{98.5}Ca_{0.5}Sr_{0.5}Zn_{0.5} alloy in Example 9 and the Mg_{98.5}Sr₁Zn_{0.5} alloy in Example 10 were manufactured by the same rapidly solidified ribbon consolidation method as the Mg₉₉₋ₓCa₁Znₓ alloys in Examples 1 to 3 except for the alloy compositions.

As illustrated in FIG. 9, it was found that the alloy in each of Examples 3, 9, and 10 had fine compounds that were homogeneously dispersed. The average grain diameter of the compounds was as fine as about 0.1 μm.

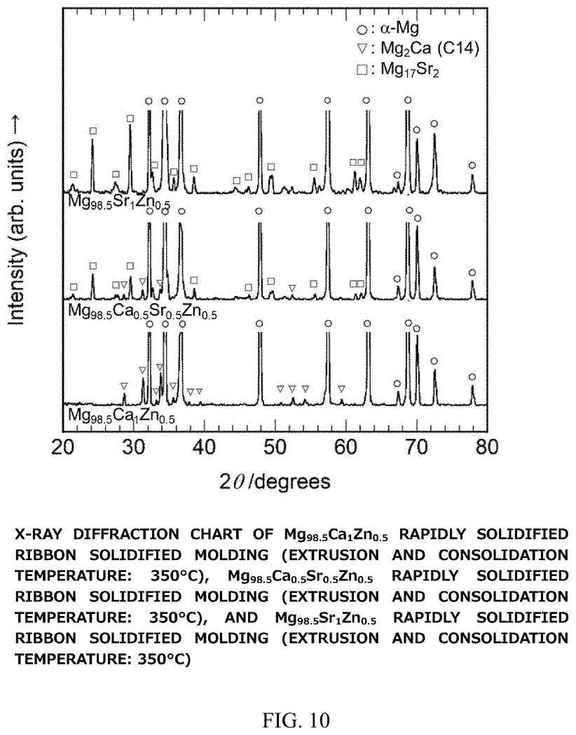

FIG. 10 is a diagram illustrating an X-ray diffraction chart of the rapidly solidified ribbon solidified molding (extrusion and consolidation temperature: 350° C.) of the Mg_{98.5}Ca₁Zn_{0.5} alloy in Example 3, the rapidly solidified ribbon solidified molding (extrusion and consolidation temperature: 350° C.) of the Mg_{98.5}Ca_{0.5}Sr_{0.5}Zn_{0.5} alloy in Example 9, and the rapidly solidified ribbon solidified molding (extrusion and consolidation temperature: 350° C.) of the Mg_{98.5}Sr₁Zn_{0.5} alloy in Example 10.

As illustrated in FIG. 10, it was confirmed that the Mg_{98.5}Ca₁Zn_{0.5} alloy in Example 3 had a compound of Mg₂Ca, the Mg_{98.5}Ca_{0.5}Sr_{0.5}Zn_{0.5} alloy in Example 9 had compounds of Mg₂Ca and Mg₁₇Sr₂, and the Mg_{98.5}Sr₁Zn_{0.5} alloy in Example 10 had a compound of Mg₁₇Sr₂. Note that, in Examples 3, 9, and 10, the production of a Mg₆Ca₂Zn₃ compound, which decreases the corrosion resistance, illustrated in FIG. 4 was not confirmed.

As the alloy in Example 11, an implant magnesium alloy was manufactured by melting and casting a Mg_{98.5}Ca₁Zn_{0.5} alloy (cooling rate: about 10 K/sec) and applying severe plastic deformation to the resultant cast material so that the average grain diameters of a-Mg grains and compounds of Mg₂Ca were decreased. ECAE was used for severe plastic deformation. Specifically, a cast of the Mg_{98.5}Ca₁Zn_{0.5} alloy as a molding material was forcedly caused to enter a molding hole having an L-shaped cross section in a molding die, and stress was applied to the cast at a 90°-bent portion of the L-shaped molding hole to obtain a solidified molding. The number of passes of ECAE is 4. The temperature during ECAE is 350° C.

In Comparative Example 4, a Mg_{98.8}Ca₁Zn_{0.2} alloy was melted and cast (cooling rate: about 10 K/sec), and the resultant cast material was extruded at a temperature of 350° C. to manufacture an extruded material.

In Comparative Example 5, a Mg_{98.5}Ca₁Zn_{0.5} alloy was melted and cast (cooling rate: about 10 K/sec), and the resultant cast material was extruded at a temperature of 350° C. to manufacture an extruded material.

In Comparative Example 6, a Mg₉₈Ca₁Zn₁ alloy was melted and cast (cooling rate: about 10 K/sec), and the resultant cast material was extruded at a temperature of 350° C. to manufacture an extruded material.

Next, tensile testing was performed on each of the alloys in Example 11 and Comparative Examples 4 to 6 under room temperature. The results are shown in Table 1. As shown in Table 1, Example 11 exhibited a tensile yield strength (yield strength) of 312 MPa and an elongation of 13.2%, thereby achieving both the tensile yield strength and the elongation.

Next, the corrosion rate of each of the alloys in Example 11 and Comparative Examples 4 to 6 in simulated body fluid was measured, and the measurement results are shown in Table 1. Note that the corrosion rate measurement method is the same as in Examples 1 to 3.

All the corrosion rates in Comparative Examples 4 to 6 were larger than 1.00 mm/year, and are indicated as “>1.00” in Table 1.

As shown in Table 1, it was found that the Mg_{98.5}Ca₁Zn_{0.5} alloy in Example 11 had a corrosion resistance higher than those in Comparative Examples 4 to 6.

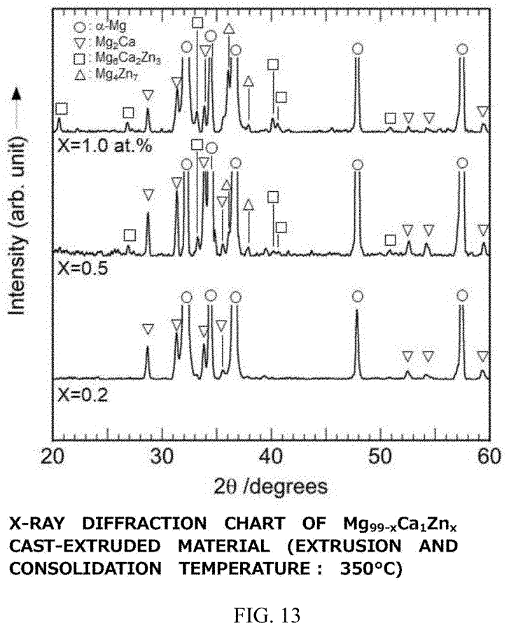

FIG. 13 is a diagram illustrating an X-ray diffraction chart (XRD) of the cast-extruded material in each of Comparative Examples 4, 5, and 6.

As illustrated in FIG. 13, in the cast-extruded materials, the compound in the Mg_{98.8}Ca₁Zn_{0.2} alloy in Comparative Example 4, which contains 0.2 at % of Zn, is only Mg₂Ca, but in the Mg_{98.5}Ca₁Zn_{0.5} alloy containing 0.5 at % of Zn in Comparative Example 5 and in the Mg₉₈Ca₁Zn₁ alloy containing 1.0 at % of Zn in Comparative Example 6, compounds of Mg₆Ca₂Zn₃ and Mg₄Zn₇ are present in addition to Mg₂Ca.

On the other hand, as shown in Table 1, in the quenched materials, only Mg₂Ca is present in Examples 1 to 8 and 11 to 13 where the alloy contains 1.0 at % or less of Zn, but a compound of Mg₆Ca₂Zn₃ other than Mg₂Ca is present in Comparative Example 2 where the alloy contains 2.0 at % of Zn and in Comparative Example 3 where the alloy contains 3.0 at % of Zn.

In other words, it was found that when the cast-extruded material contained 0.5 at % or more of Zn, a compound other than Mg₂Ca was deposited, but when the quenched material contained 2.0 at % or more of Zn, a compound other than Mg₂Ca was deposited.

From the above description, it is understood from FIG. 13 and Table 1 that, in the implant magnesium alloy, the content of Zn is 0.15 at % or more and 1.5 at % or less (preferably 0.2 at % or more and 1.0 at % or less).

Note that Table 1 shows the manufacturing method, the alloy composition, the tensile yield strength, the breaking elongation, the corrosion rate in simulated body fluid, the extrusion and consolidation temperature or cast extrusion temperature, the a-Mg grain diameter, the compound grain diameter, and the constituent phase determined by XRD (X-ray diffraction) in each of Comparative Examples 1 to 8 and Examples 1 to 14.

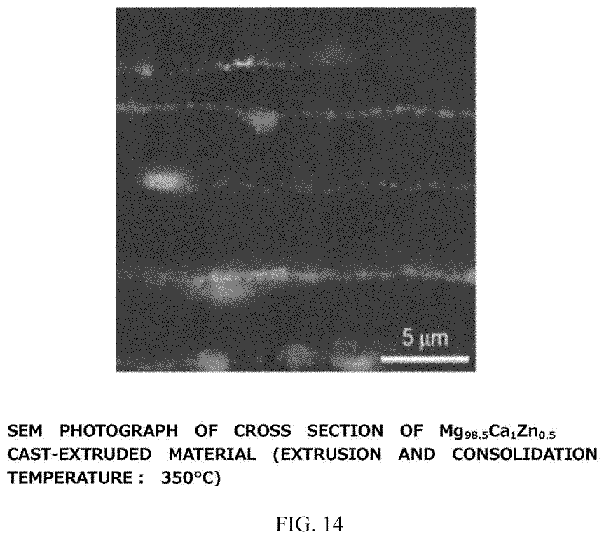

FIG. 14 is an SEM photograph of the cross section of the Mg_{98.5}Ca₁Zn_{0.5} cast-extruded material (extrusion temperature: 350° C.) in Comparative Example 5.

In the cast-extruded material in Comparative Example 5 illustrated in FIG. 14, the compounds are ununiformly dispersed, but in the rapidly solidified ribbon-extruded consolidated solidified molding illustrated in FIG. 9, the compounds are uniformly dispersed. Thus, it can be said that the rapidly solidified ribbon-extruded consolidated solidified molding is superior to the cast-extruded material in the manufacturing of a magnesium alloy having both of corrosion resistance and mechanical strength.

### EXPLANATION OF SYMBOLS

11 Single roller
12 Melting chamber
13 Roller chamber

1. An implant magnesium alloy, comprising: x at % of Zn; a total of y at % of at least one element of Ca and Sr; and the balance of Mg and inevitable impurities,
wherein x and y satisfy formulae 1 and 2 below,
0.15≤x≤1.5 (Formula 1)
0.5≤y≤1.5. (Formula 2)

2. The implant magnesium alloy according to claim 1, wherein the magnesium alloy comprises a at % of Mn, where a satisfies formula 3 below,
0.01≤a≤0.08. (Formula 3)

3. The implant magnesium alloy according to claim 1, wherein the magnesium alloy contains z at % of RE (rare-earth element), wherein z satisfies formula 4 below,
0≤z≤0.2. (Formula 4)

4. The implant magnesium alloy according to claim 1, wherein:
the magnesium alloy comprises a plurality of α-Mg grains; and
the plurality of α-Mg grains have an average grain diameter of 0.8 μm or more and 2.5 μm or less.

5. The implant magnesium alloy according to claim 1, wherein the magnesium alloy comprises at least one of compounds of Mg₂Ca and Mg₁₇Sr₂.

6. The implant magnesium alloy according to claim 5, wherein the at least one compound has an average grain diameter of 0.07 μm or more and 0.29 μm or less.

7. The implant magnesium alloy according to claim 1, wherein the magnesium alloy does not have a compound of Mg₆Ca₂Zn₃ in a constituent phase determined by X-ray diffraction.

8. The implant magnesium alloy according to claim 1, wherein the magnesium alloy does not contain an unavoidable amount or more of Al.

9. A bone fixture, comprising the implant magnesium alloy according to claim 1.

10. A method of manufacturing an implant magnesium alloy, comprising the steps of:
(a) rapidly solidifying a molten metal of a magnesium alloy to manufacture a plurality of rapidly solidified solids;
(b) solidifying the plurality of rapidly solidified solids to form a solidified molding, wherein:
the magnesium alloy comprises x at % of Zn, y at % of at least one element of Ca and Sr, and the balance of Mg and inevitable impurities; and
x and y satisfy formulae 1 and 2 below,
0.15≤x≤1.5 (Formula 1)
0.5≤y≤1.5. (Formula 2)

11. The method of manufacturing an implant magnesium alloy according to claim 10, further comprising, after the step (b), a step of extruding the solidified molding.

12. The method of manufacturing an implant magnesium alloy according to claim 10, wherein the magnesium alloy comprises a at % of Mn, and a satisfies formula 3 below,
0.01≤a≤0.08. (Formula 3)

13. The method of manufacturing an implant magnesium alloy according to claim 10, wherein the magnesium alloy comprises z at % of RE (rare-earth element), and z satisfies formula 4 below,
0≤z≤0.2. (Formula 4)

14. The method of manufacturing an implant magnesium alloy according to claim 10, wherein:
the rapidly solidified solid or the solidified molding has a plurality of α-Mg grains; and
the plurality of α-Mg grains have an average grain diameter of 0.8 μm or more and 2.5 μm or less.

15. The method of manufacturing an implant magnesium alloy according to claim 10, wherein the rapidly solidified solid or the solidified molding has at least one compound of Mg₂Ca and Mg₁₇Sr₂.

16. The method of manufacturing an implant magnesium alloy according to claim 15, wherein the at least one compound has an average grain diameter of 0.07 μm or more and 0.29 μm or less.

17. The method of manufacturing an implant magnesium alloy according to claim 10, wherein the rapidly solidified solid or the solidified molding does not have a compound of Mg₆Ca₂Zn₃ in a constituent phase determined by X-ray diffraction.

18. The method of manufacturing an implant magnesium alloy according to claim 10, wherein the magnesium alloy does not contain an unavoidable amount or more of Al.

19. A method of manufacturing a bone fixture, comprising manufacturing a bone fixture by using the method according to claim 10.

20. A method of manufacturing a bone fixture, comprising manufacturing a bone fixture by using the method according to claim 11.
